(19) United States
(12) Patent Application Publication
(10) Pub. No.: US 2025/0352122 A1
(43) Pub. Date: Nov. 20, 2025

(54) MUSCULOSKELETAL BIO-SIGNAL, POSE MONITORING SYSTEM, AND LOAD IDENTIFICATION

(71) Applicant: QATAR UNIVERSITY, Doha (QA)

(72) Inventors: Rony IBRAHIM, Doha (QA); Uvais QIDWAI, Doha (QA)

(21) Appl. No.: 19/210,612

(22) Filed: May 16, 2025

Related U.S. Application Data
(60) Provisional application No. 63/649,216, filed on May 17, 2024.

(51) Int. Cl. A61B 5/397; A61B 5/00; G16H 40/67

(52) U.S. Cl. CPC A61B 5/397; A61B 5/0004; G16H 40/67; A61B 2505/09; A61B 2562/0219

(57) ABSTRACT

Systems, methods, apparatuses, and computer program products for musculoskeletal bio-signal and pose monitoring, and muscular load detection and analysis. A method may include attaching a first sensor on a muscle belly of a person. The method may also include attaching a second sensor to a body segment of a person. The method may further include transmitting a muscle electrical activity level and motion and orientation change data to a server. The server may be configured to generate performance metrics based on the muscle electrical activity level and the motion and orientation change data

### CROSS-REFERENCE TO RELATED APPLICATION

This application claims priority from U.S. provisional patent application No. 63/649,216 filed on May 17, 2024. The contents of this earlier filed application are hereby incorporated by reference in their entirety.

### FIELD

Some example embodiments may generally relate to technologies to harvest energy. More specifically, certain example embodiments may relate to a device for musculoskeletal bio-signal and pose monitoring, and muscular load detection and analysis.

### BACKGROUND

Musculoskeletal loading monitoring involves assessing and tracking the forces and torques between internal tissues such as muscles, bones, cartilages, tendons, and ligaments. It covers variables such as joint torques, joint contact (cartilage) forces, muscle forces, and ligament forces. Musculoskeletal loading monitoring may be used to diagnose and manage conditions such as, injuries, pain, and musculoskeletal disorders. Another form of monitoring musculoskeletal health may include use of electromyography (EMG), which may provide diagnostic tests that measure electrical activity of muscles. Based on muscle electrical activity, it may be possible to provide health and functional assessments of muscles and nerves.

Muscular loading detection and analysis play a critical role in rehabilitation and training applications, particularly for athletes, stroke patients, and individuals undergoing physical therapy or strength training. Traditional methods for monitoring muscular loading often lack portability, real-time feedback, and comprehensive data analysis capabilities. Thus, there is a need for a wearable device that can accurately measure muscular loading, provide real-time feedback, and facilitate data-driven rehabilitation and training programs.

### SUMMARY

Some embodiments may be directed to a method. The method may include attaching a first sensor on a muscle belly of a person. According to certain embodiments, the first sensor is configured to determine a muscle electrical activity level from a muscle of the person. The method may also include attaching a second sensor to a body segment of a person. According to certain embodiments, the second sensor may be configured to determine a motion and orientation change data of the body segment, and the second sensor may be attached at a different location on the person compared to the first sensor. The method may further include transmitting the muscle electrical activity level and the motion and orientation change data to a server. According to certain embodiments, the server may be configured to generate performance metrics based on the muscle electrical activity level and the motion and orientation change data.

Other example embodiments may be directed to a monitoring sensor. The monitoring sensor may include means for determining a muscle electrical activity level from a muscle of a person. The monitoring sensor may also include means for determining motion and orientation change data of the person. The monitoring sensor may further include means for transmitting the muscle electrical activity level and the motion and orientation change data to a server which is configured to generate performance metrics based on the muscle electrical activity level and the motion and orientation change data.

In accordance with other example embodiments, a non-transitory computer readable medium may be encoded with instructions that may, when executed in hardware, perform a method. The method may include attaching a first sensor on a muscle belly of a person. According to certain embodiments, the first sensor is configured to determine a muscle electrical activity level from a muscle of the person. The method may also include attaching a second sensor to a body segment of a person. According to certain embodiments, the second sensor may be configured to determine a motion and orientation change data of the body segment, and the second sensor may be attached at a different location on the person compared to the first sensor. The method may further include transmitting the muscle electrical activity level and the motion and orientation change data to a server. According to certain embodiments, the server may be configured to generate performance metrics based on the muscle electrical activity level and the motion and orientation change data.

Other example embodiments may be directed to a computer program product that performs a method. The method may include attaching a first sensor on a muscle belly of a person. According to certain embodiments, the first sensor is configured to determine a muscle electrical activity level from a muscle of the person. The method may also include attaching a second sensor to a body segment of a person. According to certain embodiments, the second sensor may be configured to determine a motion and orientation change data of the body segment, and the second sensor may be attached at a different location on the person compared to the first sensor. The method may further include transmitting the muscle electrical activity level and the motion and orientation change data to a server. According to certain embodiments, the server may be configured to generate performance metrics based on the muscle electrical activity level and the motion and orientation change data.

Other example embodiments may be directed to an apparatus that may include circuitry configured to determine a muscle electrical activity level from a muscle of a person. The apparatus may also include circuitry configured to determine motion and orientation change data of the person. The apparatus may further include circuitry configured to transmit the muscle electrical activity level and the motion and orientation change data to a server which is configured to generate performance metrics based on the muscle electrical activity level and the motion and orientation change data.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 illustrates an example hardware architecture, according to certain embodiments.

FIG. 2 illustrates an example flow diagram for a data interface, according to certain embodiments.

FIG. 3A illustrates an example printed circuit board (PCB), according to certain embodiments.

FIG. 3B illustrates an example hardware assembly, according to certain embodiments.

FIG. 3C illustrates an example connection diagram for a device, according to certain embodiments.

FIG. 4 illustrates an example flow diagram of a method, according to certain embodiments.

FIG. 5 illustrates an apparatus according to certain embodiments.

### DETAILED DESCRIPTION

It will be readily understood that the components of certain example embodiments, as generally described and illustrated in the figures herein, may be arranged and designed in a wide variety of different configurations. The following is a detailed description of some embodiments of systems, methods, apparatuses, and/or computer program products for musculoskeletal bio-signal and pose monitoring, and muscular load detection and analysis.

The features, structures, or characteristics of example embodiments described throughout this specification may be combined in any suitable manner in one or more example embodiments. For example, the usage of the phrases “certain embodiments,” “an example embodiment,” “some embodiments,” or other similar language, throughout this specification refers to the fact that a particular feature, structure, or characteristic described in connection with an embodiment may be included in at least one embodiment. Thus, appearances of the phrases “in certain embodiments,” “an example embodiment,” “in some embodiments,” “in other embodiments,” or other similar language, throughout this specification do not necessarily all refer to the same group of embodiments, and the described features, structures, or characteristics may be combined in any suitable manner in one or more example embodiments.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain example embodiments, and not in limitation thereof.

Certain embodiments may provide new wearable hardware and new embedded software that provides real-time feedback about musculoskeletal loading during movement. Certain embodiments may also be applicable to athletes or individuals undergoing rehabilitation, and may be applicable for athletic training and rehabilitation. In particular, certain embodiments may provide a wireless bio-signal and pose monitoring sensor that includes an inertial measurement unit (IMU) and surface electromyography (EMG) capability. The sensor may include an accelerometer, gyroscope, magnetometer, contact surface electrodes to detect EMG bio-signal(s) generated by the muscle(s), a sensor transceiver, a voltage supply, and a microcontroller. In certain embodiments, the electrodes may be connected to the sensor via detachable wires, allowing customization of wire length and electrodes placement on the skin. The electrodes can also be connected to the sensor wirelessly. The microcontroller may be programmed to calculate the body segment orientation from fusing the measured IMU data, and the muscle activity level from detected EMG signals. These time series data may be received wirelessly by the base unit, and along with subject-specific discrete information (e.g., height, body mass, age, etc.), and fed into a machine learning model that may output musculoskeletal loading in real-time to the subject (e.g., ground reaction forces, net joint torque, net joint power, joint contact force, muscle force, tendon force, ligament tensile force, cartilage compressive stress, intervertebral disc pressure, and bone strain.). In certain embodiments, the information obtained from the sensor may be used for optimizing the prescription and monitoring of musculoskeletal rehabilitation and training.

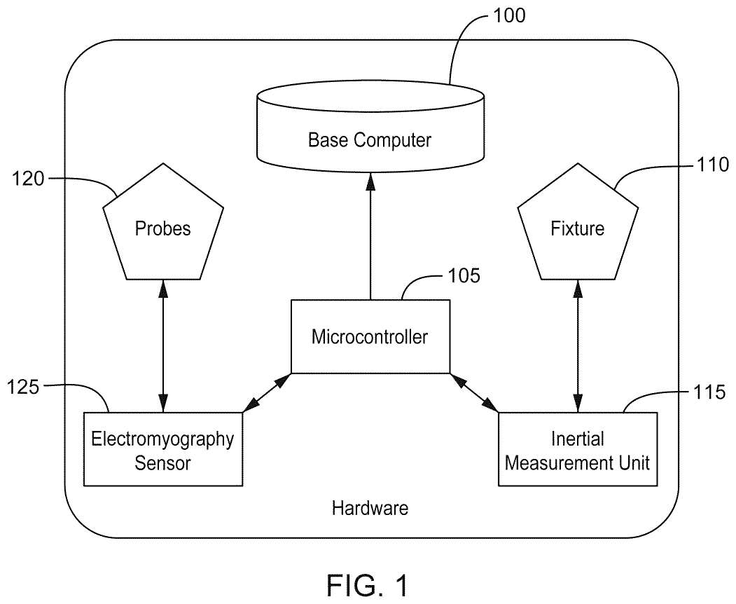

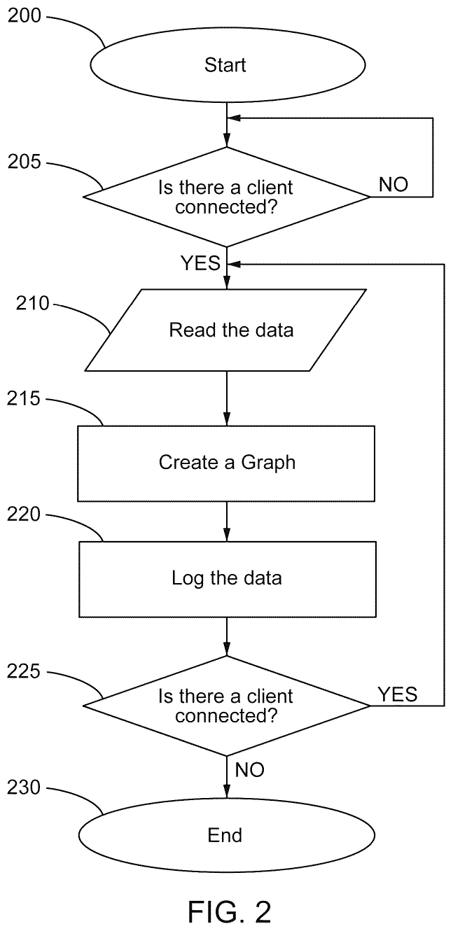

FIG. 1 illustrates an example hardware architecture, according to certain embodiments. In particular, FIG. 1 illustrates the relationship of the hardware components with each other. The microcontroller 100 (e.g., base computer) may act as a central hub for all data communication to and from the device, making it a crucial component for its overall functionality. According to certain embodiments, the components of the hardware architecture in FIG. 1 may all be part of the same device except the base computer 100. In some embodiments, the EMG 125 and IMU 115 sensors may both be connected to pins of the microcontroller 105. In certain embodiments, the IMU hardware 115 may be incorporated into a wearable device 110. From the microcontroller 105, the sensors 115, 125 may receive a power supply and allow the obtained data to be read by the microcontroller. Although one EMG sensor 125 is illustrated in FIG. 1, in other embodiments, the device may 110 may include more than one EMG sensor 125. In some embodiments, the EMG sensor 125 may be linked to the microcontroller 105 by wires of different length depending on the need or the EMG sensor 125 may be linked to the microcontroller wirelessly. According to certain embodiments, such configuration/arrangement of the components of the device 110 may allow for optimal placement for IMU and EMGs for minimum noise and measurement error/artifact. As the microcontroller 105 receives the data, it may then transmit the data to a programming platform (e.g., MATLAB) executed on a base computer 100, as illustrated in FIG. 2. The base computer 100 may receive the data and may execute an artificial intelligence (AI) model and provide results of the model to the user.

As illustrated in FIG. 1, the wearable device 110 may include inertial IMU sensors 110, 115 that may be strategically placed on a body segment to measure motion and orientation changes. The wearable device 110 may also include electromyography (EMG) sensors 120, 125 which may be placed on the muscle belly for capturing electrical activity generated during contractions. These sensors 110, 115, 120, 125 may work in tandem to provide comprehensive data on muscular loading dynamics. According to certain example embodiments, the EMG 125 and IMU 115 may be contained within the wearable device 110, and the electrodes of the EMG 125 may be attached via wires of different length (depending on the need) or wirelessly to the wearable device 110.

In certain embodiments, the IMU sensors 110, 115 may detect changes in acceleration, angular velocity, magnetic field, and orientation, providing insights into the subject's movement patterns and posture. Meanwhile, the EMG sensors 120, 125 may capture electrical signals generated by muscle contractions, offering information on muscle activation levels and fatigue.

According to some embodiments, an optimal location of the EMG electrodes 120 may be on the muscle belly, while an optimal location of the IMU sensor 110, 115 may be at a location on the body segment that has the minimum skin deformation due to muscle contraction. Conventional sensor designs restrict the user to place both sensors at the same place which harm one of the two measurements. However, the sensor of certain embodiments may integrate both sensors (e.g., IMU and EMG) but allow for flexible placement of the IMU 110, 115 and EMG 120, 125 at their individual optimal locations.

According to certain embodiments, the IMU 110, 115 and EMG sensors 120, 125, and/or a combination of IMU and EMG sensors may provide objective quantification of musculoskeletal data. According to some embodiments, the load on muscles and joints may be estimated from IMU and EMG data. Some embodiments may integrate the use of IMU 110, 115 and EMG 120, 125 sensors to provide a comprehensive assessment of muscular and articular loading. In other embodiments, the IMU 110, 115 and EMG 120, 125 sensors may be combined with detachable probes for a more thorough assessment of muscular loading during dynamic activities.

As illustrated in FIG. 1, the device may incorporate detachable or wireless EMG electrodes 120, which allows the placement of IMU 125 and EMG 115 at their optimal locations on a body segment. For instance, when measuring the orientation of the thigh and the muscle activity of the vastus medialis, existing technologies will require that the device (including the IMU) to be placed at the vastus medialis muscle belly. This makes the IMU data inaccurate because it is affected by underlying tissue movements. In contrast, the technology of certain embodiments allows the IMU 115 to be placed at the lateral aspect of the mid-thigh, and the EMG electrodes 120 to be placed at the muscle belly of the vastus medialis, which is optimal for both sensors and minimize skin artifacts and measurement error.

FIG. 2 illustrates an example server architecture for a data interface, according to certain embodiments. In some embodiments, MATLAB may implemented at a server, which may wait for a client to connect to it. Once MATLAB detects a client, it may read the data sent to it to generate a visual representation on it, and log the data it received.

As illustrated in FIG. 2, the procedure begins at 200. At 205, the server determines whether there is a client device (e.g., sensor device including, for example, IMU/EMG) is connected to the server. If the client device is not connected, the procedure returns to 205 where the server continues to determine whether a client device is connected. If the client device is connected, at 210, the server reads the data obtained from the client device, and at 215, creates a graphical representation of the data. According to certain embodiments, the collected sensor data may be wirelessly transmitted from the microcontroller of the EMG/IMU to the server. The data may include, for example, data obtained by the IMU and EMG sensors. In real-time, the subject can plot any musculoskeletal loading variable in the form of bar plot where the y-axis is the magnitude of the variable, or in the form of line plot where the y-axis is the magnitude of the variable and the x-axis is the time in seconds. Multiple plots can be created and viewed at once. The subject may also select to view the result of one variable or more in a form of dashboard, where the color of the value may change depending on its magnitude with respect to pre-set thresholds. From the graphs or the dashboard, it may be possible to determine monitor any musculoskeletal loading variable. For instance, in some embodiments, the server may execute an artificial intelligence (AI) or a machine learning (ML) model to process the received data, and fit the processed data to a muscular model to generate performance metrics such as, for example, muscle activation patterns, force exertion, fatigue levels, and movement efficiency. Various ML models that were tested with the device of certain embodiments may include, but not limited to, for example: XGBoost, Weak Ensamble Classifier, Shallow Neural Network, Support Vector Machine, Long short-term memory, and Adaptive Neuro Fuzzy Classifiers. The presented work of certain embodiments may be based on the last algorithm that worked satisfactorily with the low data volume and reasonable feature dimensionality. At 220, the sever logs/saves the data received from the IMU and EMG sensors. At 225, the server determines whether the client device is still connected to the server. If yes, the procedure returns to 210 where the server reads additional data from the IMU and EMG sensors. If the server determines that there is no client device connected, the procedure ends at 230.

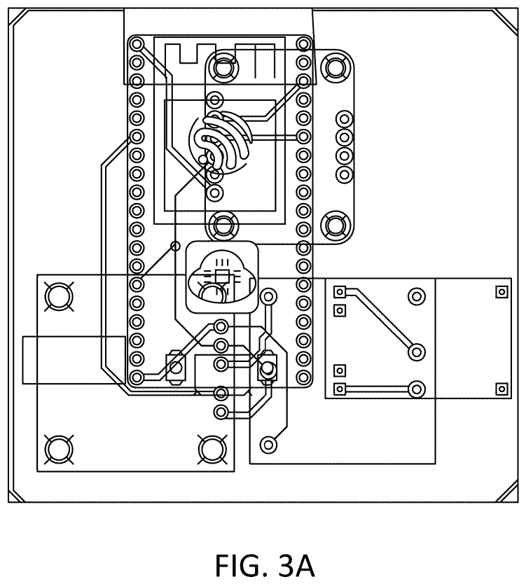

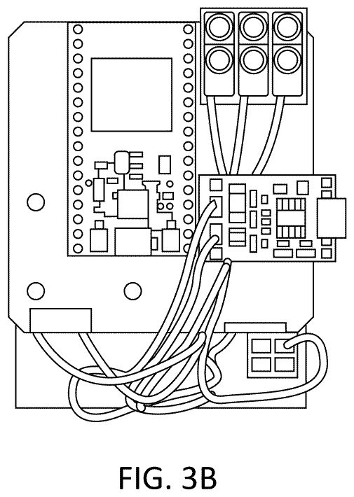

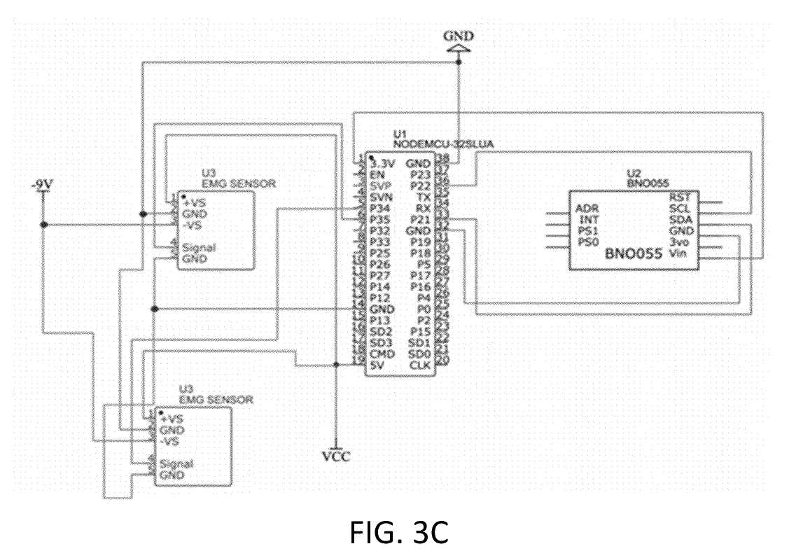

FIG. 3A illustrates an example printed circuit board (PCB), according to certain embodiments, and FIG. 3B illustrates an example hardware assembly, according to certain embodiments. For instance, FIGS. 3A and 3B illustrate the final PCB version of the connected components. Essentially, all the connections may be based on the connection diagram shown in the FIG. 3C. In particular, FIG. 3C illustrates a connection diagram for the device that corresponds to the PCN design illustrated in FIG. 3A.

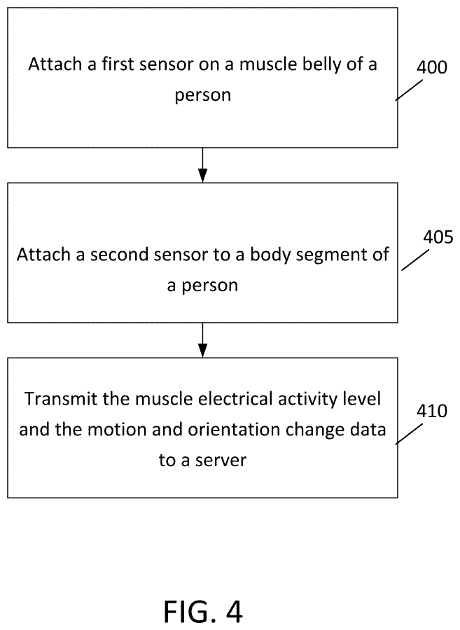

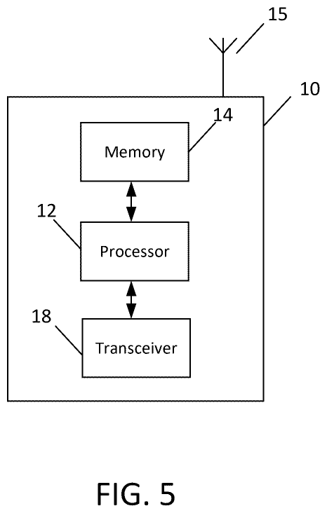

FIG. 4 illustrates an example flow diagram of a method, according to certain example embodiments. In an example embodiment, the method of FIG. 4 may be performed by a sensor device including the EMG and IMU, and a computer and/or server device similar to the apparatus 10 illustrated in FIG. 5.

According to certain example embodiments, the method of FIG. 4 may include, at 400, attaching a first sensor on a muscle belly of a person. According to certain embodiments, the first sensor may be configured to determine a muscle electrical activity level from a muscle of the person. The method may also include, at 405, attaching a second sensor to a body segment of a person. According to certain embodiments, the second sensor may be configured to determine a motion and orientation change data of the body segment, and the second sensor may be attached at a different location on the person compared to the first sensor. The method may further include, at 410, transmitting the muscle electrical activity level and the motion and orientation change data to a server. According to certain embodiments, the server may be configured to generate performance metrics based on the muscle electrical activity level and the motion and orientation change data.

According to certain embodiments, the method may also include fusing a linear acceleration, an angular velocity, and magnetic field data measured respectively by an accelerometer, a gyroscope, and magnetometer sensors inside the second sensor. According to some embodiments, the method may further include determining an orientation of the body segment in the form of quaternions. According to other embodiments, the method may also include feeding the muscle electrical activity level and the motion and orientation change data to a machine learning model.

In certain embodiments, the method may further include generating, in real time via the machine learning model, musculoskeletal loading information of the person. According to certain embodiments, the machine learning model may be trained on laboratory data where 3D kinematics were measured via optoelectronic passive marker motion capture system, 3D kinetics were measured via force platforms, and muscle activity levels were measured via EMG surface electrodes. In certain embodiments, the musculoskeletal loadings may be determined by feeding these data to a musculoskeletal model in Opensim software. After successfully training the machine learning model, it may become capable for estimating those musculoskeletal loadings calculated from laboratory data but now from field data measured from the device (IMU+EMG). Furthermore, the estimated musculoskeletal loadings may be scaled by the model to the subject characteristics by inputs such as body mass, body height, age, target movement, and external weight.

According to certain embodiments, the musculoskeletal loading information may include at least one of: muscle activation pattern; ground reaction forces; net joint torque; net joint power; joint contact force; muscle force; tendon force; ligament tensile force; cartilage compressive stress; intervertebral disc pressure; or bone strain.

FIG. 5 illustrates an apparatus 10 according to certain example embodiments. In certain example embodiments, apparatus 10 may be a sensor device, EMG, IMU, microcontroller, a computer, mobile computing device, network device, server, or other similar device. In some embodiments, apparatus 10 may be in communication (i.e., connected to either via wire or wirelessly) with other similar computer devices forming a network of connected computer devices.

In some example embodiments, apparatus 10 may include one or more processors, one or more computer-readable storage medium (for example, memory, storage, or the like), one or more radio access components (for example, a modem, a transceiver, or the like), and/or a user interface.

As illustrated in the example of FIG. 5 apparatus 10 may include or be coupled to a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. While a single processor 12 is shown in FIG. 5, multiple processors may be utilized according to other example embodiments. For example, it should be understood that, in certain example embodiments, apparatus 10 may include two or more processors that may form a multiprocessor system (e.g., in this case processor 12 may represent a multiprocessor) that may support multiprocessing. According to certain example embodiments, the multiprocessor system may be tightly coupled or loosely coupled (e.g., to form a computer cluster).

Processor 12 may perform functions associated with the operation of apparatus 10 including, as some examples, precoding of antenna gain/phase parameters, encoding and decoding of individual bits forming a communication message, formatting of information, and overall control of the apparatus 10, including processes illustrated in FIGS. 1-4.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RAM), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform tasks as described herein.

In certain example embodiments, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10 to perform any of the methods illustrated in FIGS. 1-4.

In some example embodiments, apparatus 10 may also include or be coupled to one or more antennas 15 for receiving a downlink signal and for transmitting via an uplink from apparatus 10. Apparatus 10 may further include a transceiver 18 configured to transmit and receive information. The transceiver 18 may also include a radio interface (e.g., a modem) coupled to the antenna 15. The radio interface may include other components, such as filters, converters signal shaping components, and the like, to process symbols, carried by a downlink or an uplink.

For instance, transceiver 18 may be configured to modulate information on to a carrier waveform for transmission by the antenna(s) 15 and demodulate information received via the antenna(s) 15 for further processing by other elements of apparatus 10. In other example embodiments, transceiver 18 may be capable of transmitting and receiving signals or data directly. Additionally or alternatively, in some example embodiments, apparatus 10 may include an input and/or output device (I/O device). In certain example embodiments, apparatus 10 may further include a user interface, such as a graphical user interface or touchscreen.

In certain example embodiments, memory 14 stores software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software.

According to certain example embodiments, processor 12 and memory 14 may be included in or may form a part of processing circuitry or control circuitry. In addition, in some example embodiments, transceiver 18 may be included in or may form a part of transceiving circuitry.

As used herein, the term “circuitry” may refer to hardware-only circuitry implementations (e.g., analog and/or digital circuitry), combinations of hardware circuits and software, combinations of analog and/or digital hardware circuits with software/firmware, any portions of hardware processor(s) with software (including digital signal processors) that work together to cause an apparatus (e.g., apparatus 10) to perform various functions, and/or hardware circuit(s) and/or processor(s), or portions thereof, that use software for operation but where the software may not be present when it is not needed for operation. As a further example, as used herein, the term “circuitry” may also cover an implementation of a hardware circuit or processor (or multiple processors), or portion of a hardware circuit or processor, and its accompanying software and/or firmware.

In certain example embodiments, apparatus 10 may be controlled by memory 14 and processor 12 to determine a muscle electrical activity level from a muscle of a person. Apparatus 10 may also be controlled by memory 14 and processor 12 to determine motion and orientation change data of the person. Apparatus 10 may further be controlled by memory 14 and processor 12 to transmit the muscle electrical activity level and the motion and orientation change data to a server which is configured to generate performance metrics based on the muscle electrical activity level and the motion and orientation change data.

In some embodiments, an apparatus (e.g., apparatus 10) may include means for performing a method, a process, or any of the variants discussed herein. Examples of the means may include one or more processors, memory, controllers, transmitters, receivers, sensors, and/or computer program code for causing the performance of the operations.

Certain embodiments may further be directed to an apparatus that includes means for performing any of the methods described herein including, for example, means for determining a muscle electrical activity level from a muscle of a person. The apparatus may also include means for determining motion and orientation change data of the person. The apparatus may further include means for transmitting the muscle electrical activity level and the motion and orientation change data to a server which is configured to generate performance metrics based on the muscle electrical activity level and the motion and orientation change data.

Certain embodiments described herein provide several technical improvements, enhancements, and/or advantages. In some embodiments, it may be possible to provide exceptional portability such that the wearable device may allow for continuous monitoring of musculoskeletal loading during rehabilitation and training activities, even in real-world environments. Musculoskeletal loading variables include: ground reaction forces, net joint torque, net joint power, joint contact force, muscle force, tendon force, ligament tensile force, cartilage compressive stress, intervertebral disc pressure, and bone strain. The IMU senor and EMG electrodes may be placed at their individual optimal location(s) on the body segment in-motion. The device may also provide real-time feedback to the subject, enabling adjustments to technique and intensity during exercises. In some embodiments, the device may utilize AI-based models to analyze sensor data, and generate actionable insights for optimizing rehabilitation and training programs. Some embodiments of the device may also provide versatility suitable for use by athletes, patients, and individuals undergoing physical therapy or strength training.

According to other embodiments, the wearable device for musculoskeletal loading detection and analysis may provide significant advancement in rehabilitation and training technology. For example, by integrating IMU and EMG sensors with detachable or wireless electrodes and AI-based analysis capabilities, the device may offer a comprehensive solution for monitoring and optimizing muscular activity and the load of the joint/tissues. As such, it may be possible to improve outcomes for athletes and patients alike.

Other embodiments may provide real-time feedback which can enable providers to provide immediate feedback to the subject, enabling adjustments to technique, intensity, modality, and type of exercises.

As described herein, a computer program product may include one or more computer-executable components which, when the program is run, are configured to carry out some example embodiments. The one or more computer-executable components may be at least one software code or portions of it. Modifications and configurations required for implementing functionality of certain example embodiments may be performed as routine(s), which may be implemented as added or updated software routine(s). Software routine(s) may be downloaded into the apparatus.

As an example, software or a computer program code or portions of code may be in a source code form, object code form, or in some intermediate form, and may be stored in some sort of carrier, distribution medium, or computer readable medium, which may be any entity or device capable of carrying the program. Such carriers may include a record medium, computer memory, read-only memory, photoelectrical and/or electrical carrier signal, telecommunications signal, and software distribution package, for example. Depending on the processing power needed, the computer program may be executed in a single electronic digital computer or it may be distributed amongst a number of computers. The computer readable medium or computer readable storage medium may be a non-transitory medium.

In other example embodiments, the functionality may be performed by hardware or circuitry included in an apparatus (e.g., apparatus 10), for example through the use of an application specific integrated circuit (ASIC), a programmable gate array (PGA), a field programmable gate array (FPGA), or any other combination of hardware and software. In yet another example embodiment, the functionality may be implemented as a signal, a non-tangible means that can be carried by an electromagnetic signal downloaded from the Internet or other network.

According to certain example embodiments, an apparatus, such as a node, device, or a corresponding component, may be configured as circuitry, a computer or a microprocessor, such as single-chip computer element, or as a chipset, including at least a memory for providing storage capacity used for arithmetic operation and an operation processor for executing the arithmetic operation.

One having ordinary skill in the art will readily understand that the invention as discussed above may be practiced with steps in a different order, and/or with hardware elements in configurations which are different than those which are disclosed. Therefore, although the invention has been described based upon these example embodiments, it would be apparent to those of skill in the art that certain modifications, variations, and alternative constructions would be apparent, while remaining within the spirit and scope of example embodiments.

We claim:

1. A monitoring sensor, comprising:
at least one processor; and
at least one memory storing instructions which, when executed by the at least one processor, cause the apparatus to at least:
determine a muscle electrical activity level from a muscle of a person;
determine motion and orientation change data of the person; and
transmit the muscle electrical activity level and the motion and orientation change data to a server which is configured to generate performance metrics based on the muscle electrical activity level and the motion and orientation change data.

2. The monitoring sensor according to claim 1, wherein the muscles electrical activity level and the motion and orientation change data are determined at different locations on the person.

3. The monitoring sensor according to claim 1, wherein the muscle electrical activity level is determined on a muscle belly of the person.

4. The monitoring sensor according to claim 1, wherein the motion and orientation change data comprises at least one of the following:
linear acceleration,
angular velocity, magnetic field, or
orientation in form of quaternions.

5. The monitoring sensor according to claim 1, wherein the muscle electrical activity level comprises electrical activity generated by muscle contractions of the person.

6. The monitoring sensor according to claim 1, wherein the determination of the motion and orientation change data is based on a linear and an angular motion of the body segment to which the monitoring sensor is attached.

7. The monitoring sensor according to claim 1, wherein the performance metrics comprise at least one of the following:
muscle activation pattern,
ground reaction forces,
net joint torque,
net joint power,
joint contact force,
muscle force,
tendon force,
ligament tensile force,
cartilage compressive stress,
intervertebral disc pressure, or
bone strain.

8. A method of monitoring musculoskeletal function, comprising:
attaching a first sensor on a muscle belly of a person, wherein the first sensor is configured to determine a muscle electrical activity level from a muscle of the person;
attaching a second sensor to a body segment of a person, wherein the second sensor is configured to determine a motion and orientation change data of the body segment, and wherein the second sensor is attached at a different location on the person compared to the first sensor; and
transmitting the muscle electrical activity level and the motion and orientation change data to a server, wherein the server is configured to generate performance metrics based on the muscle electrical activity level and the motion and orientation change data.

9. The method of monitoring musculoskeletal function according to claim 8, further comprising:
fusing a linear acceleration, an angular velocity, and magnetic field data measured respectively by an accelerometer, a gyroscope, and magnetometer sensors inside the second sensor; and
determining an orientation of the body segment in the form of quaternions.

10. The method of monitoring musculoskeletal function according to claim 8, further comprising:
feeding the muscle electrical activity level and the motion and orientation change data to a machine learning model; and
generating, in real time via the machine learning model, musculoskeletal loading information of the person.

11. The method of monitoring musculoskeletal function according to claim 10, wherein the musculoskeletal loading information comprises at least one of:
muscle activation pattern,
ground reaction forces,
net joint torque,
net joint power,
joint contact force,
muscle force,
tendon force,
ligament tensile force,
cartilage compressive stress,
intervertebral disc pressure, or
bone strain.

12. A non-transitory computer readable medium encoded with instructions that, when executed in hardware, perform a process, the process comprising:
determining, by a first sensor, a muscle electrical activity level from a muscle of the person;
determining, by a second sensor, a motion and orientation change data of the body segment, and wherein the second sensor is attached at a different location on the person compared to the first sensor; and
transmitting the muscle electrical activity level and the motion and orientation change data to a server, wherein the server is configured to generate performance metrics based on the muscle electrical activity level and the motion and orientation change data.

13. The non-transitory computer readable medium according to claim 12, wherein the instructions, when executed by the at least one processor, further cause the apparatus to perform:
fusing a linear acceleration, an angular velocity, and magnetic field data measured respectively by an accelerometer, a gyroscope, and magnetometer sensors inside the second sensor; and
determining an orientation of the body segment in the form of quaternions.

14. The non-transitory computer readable medium according to claim 12, wherein the instructions, when executed by the at least one processor, further cause the apparatus to perform:
feeding the muscle electrical activity level and the motion and orientation change data to a machine learning model; and
generating, in real time via the machine learning model, musculoskeletal loading information of the person.

15. The non-transitory computer readable medium according to claim 14, wherein the musculoskeletal loading information comprises at least one of:
muscle activation pattern,
ground reaction forces,
net joint torque,
net joint power,
joint contact force,
muscle force,
tendon force,
ligament tensile force,
cartilage compressive stress,
intervertebral disc pressure, or
bone strain.
